(19) United States
(12) Patent Application Publication
(10) Pub. No.: US 2026/0207207 A1
(43) Pub. Date: Jul. 23, 2026

(54) Surgical Screws and Tools for Insertion

(71) Applicant: Tyber Medical LLC, Bethlehem, PA (US)

(72) Inventors: Leo Roux, Caluire-et-Cuire (FR); Yoann Taymans, Meyzieu (FR); Daniel Jacob Zimmerman, Wynnewood, PA (US); David Hannah, Nazareth, PA (US)

(21) Appl. No.: 19/567,215

(22) Filed: Mar. 15, 2026

Related U.S. Application Data
(63) Continuation of application No. PCT/US24/46060, filed on Sep. 11, 2024.
(60) Provisional application No. 63/538,523, filed on Sep. 15, 2023.
(60) Provisional application No. 63/546,018, filed on Oct. 27, 2023.
(60) Provisional application No. 63/575,282, filed on Apr. 5, 2024.

(51) Int. Cl. A61B 17/17

(52) U.S. Cl. CPC A61B 17/1775; A61B 17/1717

(57) ABSTRACT

A targeting guide includes a body having a distal end. The distal end has a pusher with a threaded knob such that the threaded knob is rotatable to extend or retract a pusher plate. A first K-wire guide has a sleeve at a distal end thereof. The sleeve has at least two through-openings sized to allow the insertion of a K-wire through each of the at least two through-openings.

### BACKGROUND OF THE INVENTION

### Field of the Invention

The invention relates to screws that can be used with a beveled head to reduce

### Description of the Related Art

Screws can be implanted into bone to repair broken bones. Different screws are used depending on the location of the break, the severity of the break, and the bone(s) that is/are being repaired.

It can be advantageous to angle the screw at an oblique angle relative to the axis of the bone to obtain proper connection between adjacent bones or bone fragments. This, however, can result in at least a part of the screw head extending above the bone, which can lead to undesired bumps along the bone surface and the potential for the screw head to tear into the skin, possibly resulting in infection.

It would be beneficial to provide a screw that extends outside of the bone as little as possible and also provides a rounded surface to reduce unwanted bumps and potential skin tear sites.

### SUMMARY OF THE INVENTION

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

In one embodiment, the present invention is an intramedullary screw that includes an elongate threaded shaft having a proximal end and a distal end, and a central longitudinal axis extending between the proximal end and the distal end, the shaft being fully cannulated. A cannulation extends along the longitudinal axis between the proximal end and the distal end. A head is located at the proximal end and has a larger outer diameter than the shaft. The head has a pocket configured to releasably accept a driver inserted therein. A cutting tip is located at the distal end.

### BRIEF DESCRIPTION OF THE DRAWINGS

The accompanying drawings, which are incorporated herein and constitute part of this specification, illustrate the presently preferred embodiments of the invention, and, together with the general description given above and the detailed description given below, serve to explain the features of the invention. In the drawings:

FIG. 1 is a side elevational view of an exemplary embodiment of a beveled screw according to the present invention;

FIG. 2 is a front elevational view of the screw of FIG. 1;

FIG. 3 is an enlarged side elevational view of the top portion of the screw of FIG. 1;

FIG. 4 is an enlarged perspective view of the head of the screw of FIG. 1;

FIG. 5 is a sectional view of the screw of FIG. 1 implanted into a bone

FIG. 6 is a is a side elevational view of an intramedullary contact pitch screw according to an alternative exemplary embodiment of the present invention;

FIG. 7 is a side elevational view of the screw of fig. 6, with the threads removed;

FIG. 8 is a sectional view of the screw of FIG. 1, taken along lines 8-8 of FIG. 6;

FIG. 9 is an enlarged portion of the thread of FIG. 6, taken along circle 9 of FIG. 6;

FIG. 10 is a proximal end elevational view of the screw of FIG. 6

FIG. 11 is a side elevational view of the screw of FIG. 6;

FIG. 12 is a perspective view of a distal end of the screw of FIG. 11;

FIG. 13 is a perspective view of the proximal end of the screw of FIG. 12, with the screw rotated;

FIG. 14 is a side elevational view of an alternative embodiment of a screw;

FIG. 15 is a perspective view of a distal end of the screw of FIG. 14;

FIG. 16 is a perspective view of the proximal end of the screw of FIG. 15, with the screw rotated;

FIG. 17 is a side elevational view of an intramedullary screw according to an exemplary embodiment of the present invention;

FIG. 18 is a blank used to form the screw of FIG. 17;

FIG. 19 is a sectional view of the screw of FIG. 17, taken along lines 19-19 of FIG. 17;

FIG. 20 is a proximal end elevational view of the screw of FIG. 17;

FIG. 21 is a distal end elevational view of the screw of FIG. 17;

FIG. 22 is an enlarged view of detail 22 of FIG. 19;

FIG. 23 is a first perspective view of a targeting guide according to a first exemplary embodiment of the present invention;

FIG. 24 is a second perspective view of the targeting guide of FIG. 23;

FIG. 25 is a first perspective view of a targeting guide according to a second exemplary embodiment of the present invention;

FIG. 26 is a second perspective view of the targeting guide of FIG. 25;

FIG. 27 is a first perspective view of a targeting guide according to a third exemplary embodiment of the present invention;

FIG. 28 is a second perspective view of the targeting guide of FIG. 27;

FIG. 29 is a first perspective view of a targeting guide according to a fourth exemplary embodiment of the present invention;

FIG. 30 is a second perspective view of the targeting guide of FIG. 29;

FIG. 31 is a perspective view of a targeting guide according to a fifth exemplary embodiment of the present invention;

FIG. 32 is a perspective view of the targeting guide of FIG. 31 being attached to a metatarsal;

FIG. 33 is a perspective view of a K-wire being inserted into the guide of FIG. 31 and into the distal portion of the metatarsal of FIG. 32;

FIG. 34 is a perspective view of the guide of FIG. 33 with K-wire guides being inserted into the guide and into the proximal portion of the metatarsal of FIG. 32;

FIG. 35 is a perspective view of K-wires being inserted though the K-wire guides of FIG. 34 and into the distal and proximal portions of the metatarsal of FIG. 34;

FIG. 36 is a perspective view of the guide of FIG. 35 with the K-wire guides having been removed;

FIG. 37 is a perspective view of the guide of FIG. 36 with the K-wire removed from the distal portion of the metatarsal of FIG. 33; and

FIG. 38 is a perspective view of the K-wires of FIG. 35 with screws inserted over the K-wires, through the proximal portion of the metatarsal and into the distal portion of the metatarsal.

### DETAILED DESCRIPTION

In the drawings, like numerals indicate like elements throughout. Certain terminology is used herein for convenience only and is not to be taken as a limitation on the present invention. The terminology includes the words specifically mentioned, derivatives thereof and words of similar import. The embodiments illustrated below are not intended to be exhaustive or to limit the invention to the precise form disclosed. These embodiments are chosen and described to best explain the principle of the invention and its application and practical use and to enable others skilled in the art to best utilize the invention.

Reference herein to “one embodiment” or “an embodiment” means that a particular feature, structure, or characteristic described in connection with the embodiment can be included in at least one embodiment of the invention. The appearances of the phrase “in one embodiment” in various places in the specification are not necessarily all referring to the same embodiment, nor are separate or alternative embodiments necessarily mutually exclusive of other embodiments. The same applies to the term “implementation.”

As used in this application, the word “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion.

The word “about” is used herein to include a value of +/−10 percent of the numerical value modified by the word “about” and the word “generally” is used herein to mean “without regard to particulars or exceptions.”

Additionally, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

Unless explicitly stated otherwise, each numerical value and range should be interpreted as being approximate as if the word “about” or “approximately” preceded the value of the value or range.

The use of figure numbers and/or figure reference labels in the claims is intended to identify one or more possible embodiments of the claimed subject matter in order to facilitate the interpretation of the claims. Such use is not to be construed as necessarily limiting the scope of those claims to the embodiments shown in the corresponding figures.

It should be understood that the steps of the exemplary methods set forth herein are not necessarily required to be performed in the order described, and the order of the steps of such methods should be understood to be merely exemplary. Likewise, additional steps may be included in such methods, and certain steps may be omitted or combined, in methods consistent with various embodiments of the present invention.

Although the elements in the following method claims, if any, are recited in a particular sequence with corresponding labeling, unless the claim recitations otherwise imply a particular sequence for implementing some or all of those elements, those elements are not necessarily intended to be limited to being implemented in that particular sequence.

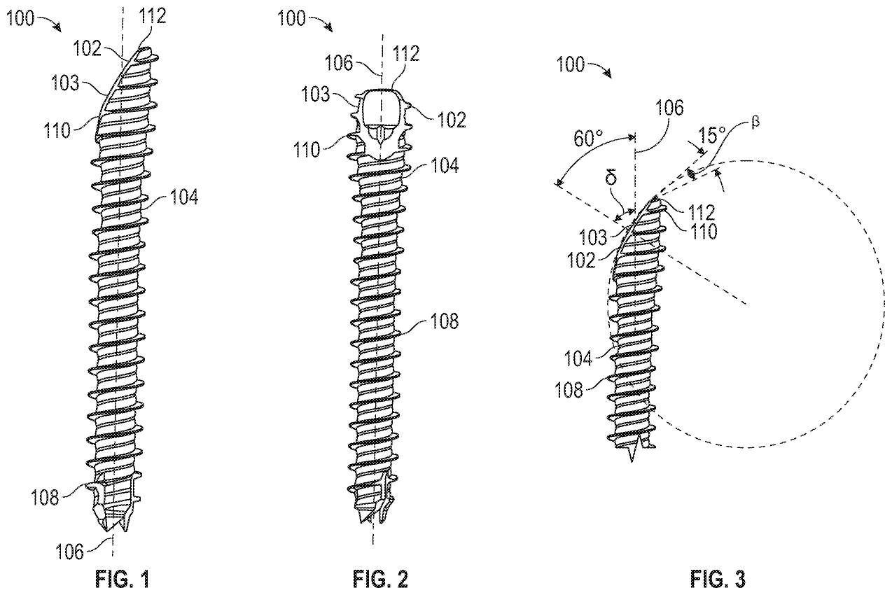

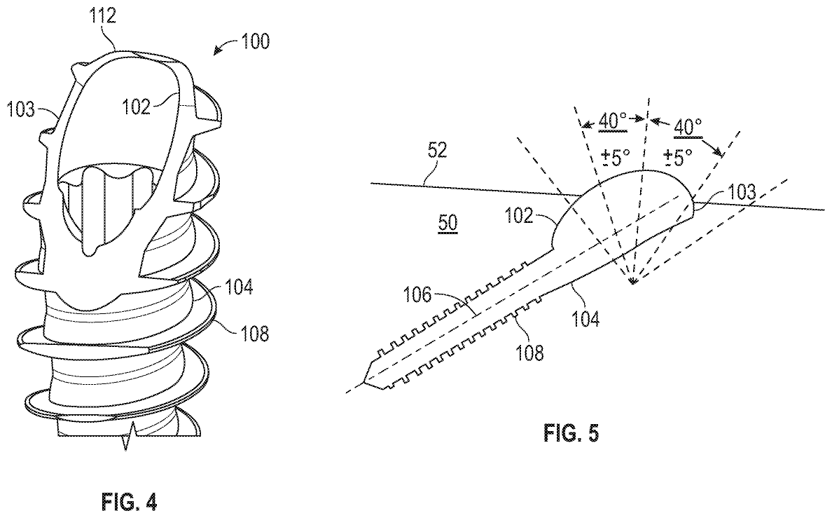

Referring to the Figures, a beveled screw 100 according to an exemplary embodiment of the present invention is shown. Screw 100 includes a screw head 102 with a bevel 103. Screw head 102 is attached to an elongate shaft 104 having a central longitudinal axis 106. Screw head 102 includes threads 108 and, as can be seen in FIGS. 1-4, a top end 110 of thread 108 terminates at bevel 103. Thread 108 can be continuous or, alternatively, thread 108 can be discontinuous around head 102.

Screw head 102 forms a dome shape in a plane extending along the line defining bone surface 52 and extending perpendicularly from the plane of the paper. Screw head 102 is not beveled with a straight cut, but instead arcs with a constant radius of curvature, forming the dome shape. In an exemplary embodiment, the radius can be between about 5 mm and about 15 mm.

A trailing, or proximal end 112 of head 102 spans an arc of about 15 degrees to minimize or eliminate proximal end 112 of head 102 extending outwardly from bone 50 after implantation.

Because screw head 102 is dome-shaped, screw head 102 is also elliptical in shape and gives the surgeon a little bit of wiggle room so if the insertion angle is not perfect, screw head 102 will still have a smooth transition at implantation. The present design makes the elliptical shape larger and more forgiving to allow the surgeon to focus on where the distal end of the screw 100 needs to be without worrying too much about the proximal end(screw head 102) being smooth/flush with bone surface 52 of bone 50. As shown in FIG. 5, the screw 100 anticipates an insertion angle of 40 degrees from the surface, with a tolerance of about ±5 degrees so that the screw 100 is more forgiving for different anatomies. While the anticipated insertion angle is 40 degrees, those skilled in the art will recognize that modifications to screw head 102 for insertion angles different from 40 degrees are also contemplated in this application.

This specified angle ensures the rounded screw head 102 will be flush with the bone surface 52 after implantation, providing a smooth transition to prevent or reduce skin irritation.

Referring specifically to FIG. 3, angle δ is the axis of revolution that forms the dome shape and angle β is the resultant of this dome from its center to its ends. Angle β allows screw 100 to follow an erroneous trajectory without allowing sharp edges of screw 100 to protrude from the bone. In an exemplary embodiment, angle δ can be about 60 degrees +/−5 degrees and angle β can be 15 degrees +/−5 degrees.

Screw 100 can be a fully threaded screw, as shown in FIGS. 1-5. Alternatively, screw 100 can be a compressive screw with threads that extend only part way from head 102 and part way from the tip, with central part of shaft 104 being threadless.

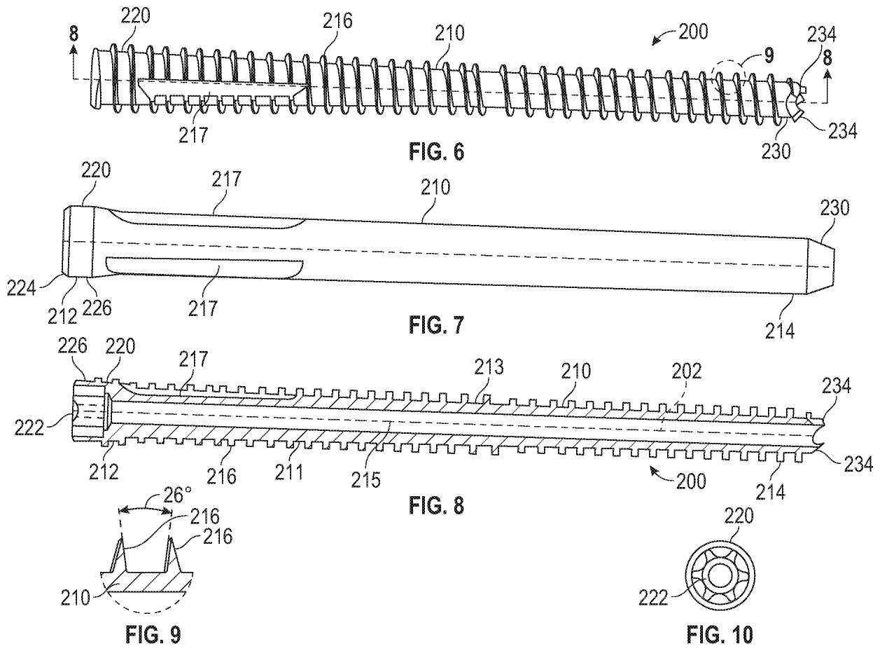

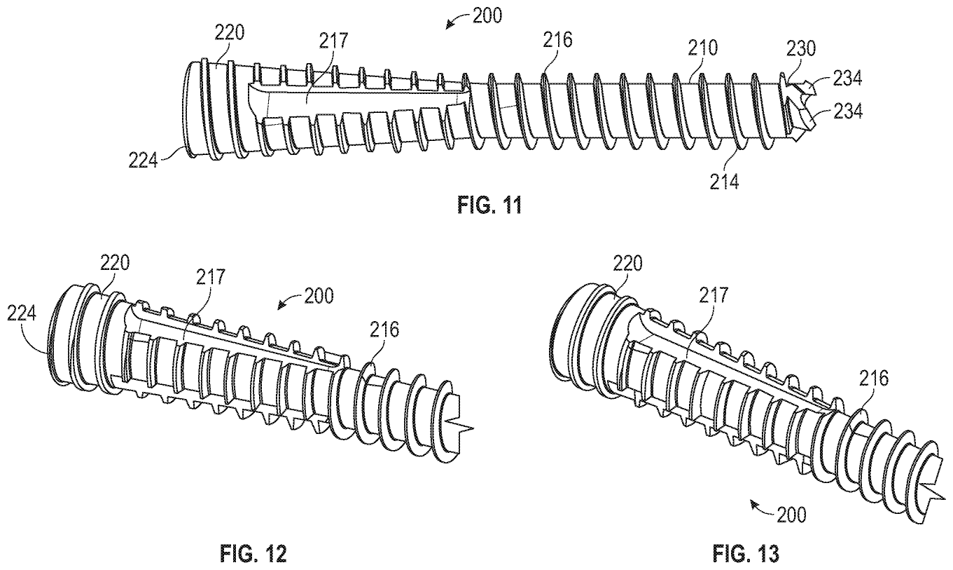

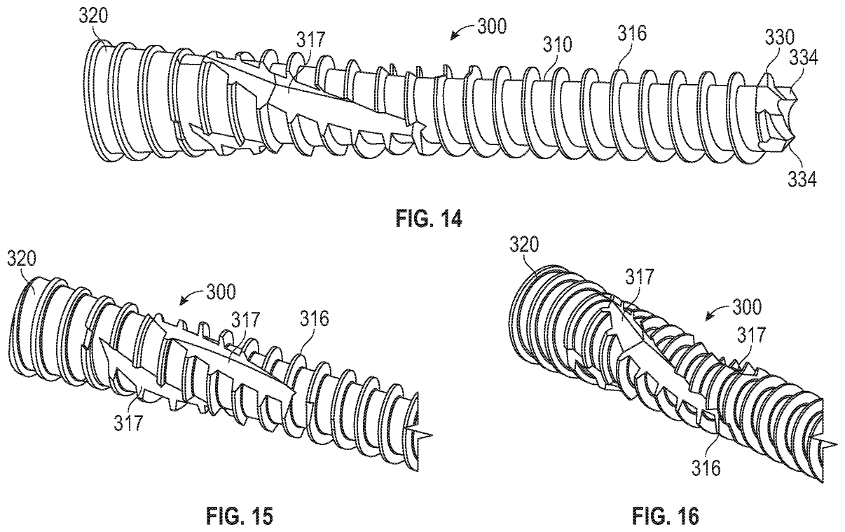

Referring to FIGS. 6-13, a constant pitch intramedullary screw 200 is shown. Screw 200 can be used to fix, among other things, phalangeal and metacarpal fractures. Exemplary, but not limiting, dimensions of screw 200 are shown in FIGS. 6-9.

Screw 200 includes an elongate shaft 210 having a proximal end 212 and a distal end 214, and a central longitudinal axis 202 extending between the proximal end 212 and the distal end 214. Shaft 210 is fully cannulated, with a cannulation 215 extending along the longitudinal axis between the proximal end 212 and the distal end 214. Cannulation 215 in shaft 210 is sized to allow a guide wire (not shown) to extend therethrough. By way of example only, screw 200 can be provided in various lengths, depending on the required usage, and can extend between about 40 mm and about 70 mm in length.

Shaft 210 has a proximal portion 211 and a distal portion 213. Shaft 210 includes a plurality of flutes 217 formed along an exterior of shaft 210 within proximal portion 211. In an exemplary embodiment, three flutes 217 are evenly spaced 120 degrees apart from each other around the exterior of shaft 210. Flutes 217 are provided to reduce the insertion torque. The proximal portion 211 of shaft 210 has a larger diameter than distal portion 213, therefore the torque will start to increase due to friction as screw 200 is advanced into bone. This increased torque can cause the bone to start to rotate or could break a screwdriver or screw 200. The flutes 217 are tapping the bone to reduce that torque. A thread 216 extends along the shaft 210. In an exemplary embodiment, only a single thread 216 is provided. Those skilled in the art, however, will recognize that more than a single thread 216 can be provided. Thread 216 is broken at each flute 217, but continues across flute 217 as if flute 217 was not present and thread 216 was continuous.

Thread 216 has a constant pitch. In an exemplary embodiment, thread 216 has a 26 degree pitch angle between adjacent thread portions. See specifically FIG. 9.

Thread 216 has a tapered minor diameter along proximal portion 211 of shaft 210 and a straight minor diameter along distal portion 213 of shaft 210.

Screw 200 further includes a head 220 located at the proximal end 212. The head 220 has a larger outer diameter than the shaft 210. The head 220 has a pocket 222 that is configured to releasably accept a driver (not shown) inserted therein. The driver can be a hexalobe driver, a hex head driver, or other suitable driver. See FIG. 10. Thread 216 extends along the head 220 and has a tapered minor diameter that continues the tapered minor diameter along proximal portion 211 of shaft 210.

In an exemplary embodiment, head 220 tapers outwardly from the shaft 210 at a 10 degree angle. Those skilled in the art, however, will recognize that head 220 can taper outwardly from shaft 210 at more or less than a 10 degree angle. Tapered head 220 continues the taper from proximal portion 211 of shaft 210.

A proximal end 224 of the head 220 has a beveled surface. An intermediate part 226 of the head 220 between the 10 degree angle and the beveled surface of proximal end 224 has a constant outer diameter.

A cutting tip 230 is located at the distal end 214 and has a plurality of cutting flutes 234 formed therein. In an exemplary embodiment, three cutting flutes 234 are provided. Cutting flutes 234 each have a 45 degree cutting angle, although those skilled in the art will recognize that the cutting flutes can have a larger or smaller than 45 degree cutting angle.

FIGS. 14-16 show a screw 300, similar to screw 200, but with helical flutes 317. Flutes 317 begin toward proximal end 320 and extend along shaft 310 in the same direction as threads 316 toward distal end 340 and cutting flutes 334. In an exemplary embodiment, three flutes 317 are evenly spaced around shaft 310 and wind around a 120 degree arc around the exterior of shaft 310, such that, when one flute 317 ends at its distal end, an adjacent flute 317 begins at the proximal end 320 at the same radial location.

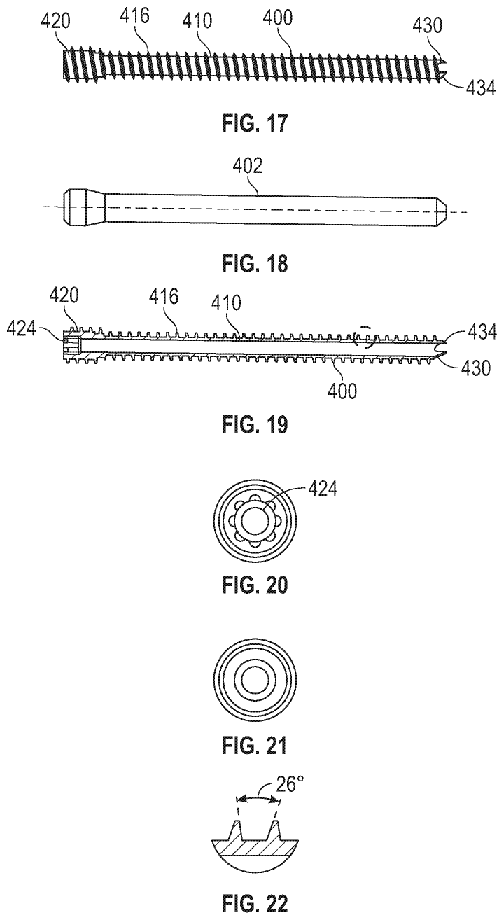

FIGS. 17-22 show a screw 400 that can be used to fix metacarpal fractures. Screw 400 is similar to screw 200, but without flutes 217. Screw 400 is formed from a blank 402 and includes a shaft 410 having a proximal end 420 with a proximal tip 224 and a distal end 430 with distal cutting flutes 434. A single thread 416 extends the length of screw 400, although those skilled in the art will recognize that multiple threads 416 can be provided.

Those skilled in the art will recognize that the heads of screws 300, 400 can be rounded similarly to the head 102 in screw 100 described above.

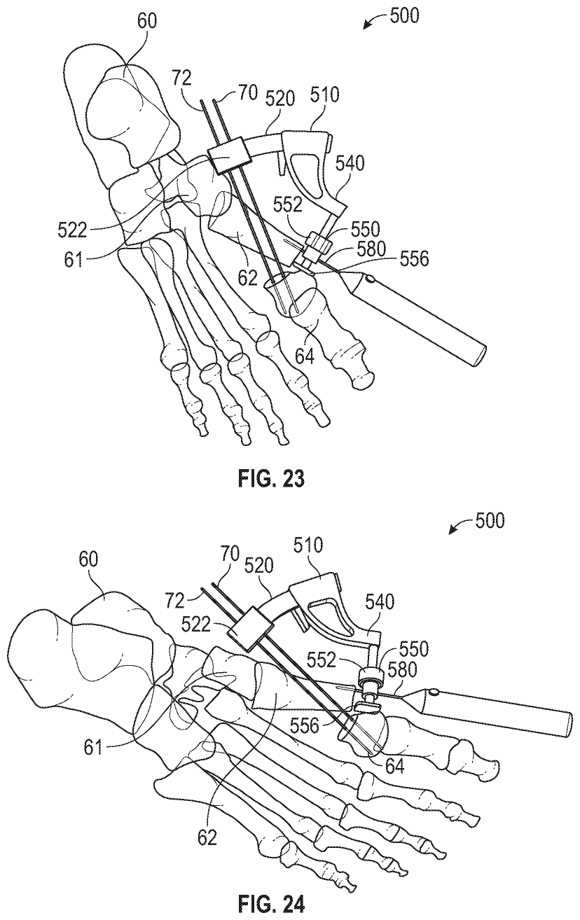

Referring now to FIGS. 23 and 24, a metatarsal screw guide 500 (“guide 500”) according to an exemplary embodiment of the invention is shown. Guide 500 is used to align and connect a proximal part of a metatarsal 62 and a distal part of the metatarsal 64 on a foot 60 after a procedure, such as a bunionectomy. Guide 500 has a body 510 and an adjustable arcuate proximal K-wire guide 520 having a sleeve 522 that guides at least two K-wires 70, 72 into distal part of metatarsal 64.

Body 510 has a distal end 540 having a pusher 550 with a threaded knob 552 that can be rotated to extend or retract a pusher plate 556 to displace distal part of metatarsal 64 relative to proximal part of metatarsal 62 so that a drill 580 can be inserted into a distal end of the proximal part of metatarsal 62.

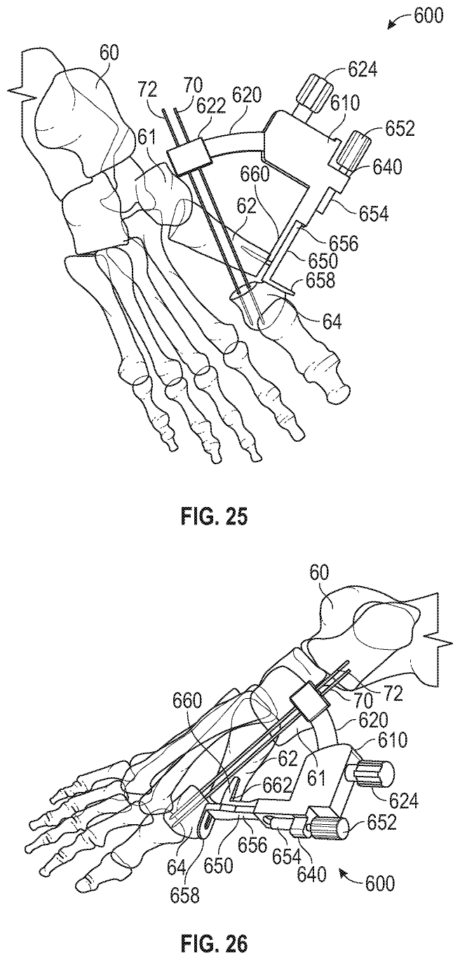

Referring to FIGS. 25 and 26, a metatarsal screw guide 600 (“guide 600”) according to an alternative exemplary embodiment of the invention is shown. Guide 600 is used to align and connect proximal part of metatarsal 62 and distal part of the metatarsal 64 on foot 60 after a procedure, such as a bunionectomy. Guide 600 has a body 610 and an adjustable arcuate proximal K-wire guide 620 having a sleeve 622 that guides at least two K-wires 70, 72 into distal part of metatarsal 64. A locking knob 624 can be rotated to releasably lock K-wire guide 620 relative to body 610.

Body 610 has a distal end 640 having a pusher 650 with a threaded knob 652 having a threaded portion 654 that engages a complementary thread 656 on pusher 650 to extend or retract a pusher plate 658 to displace distal part of metatarsal 64 relative to proximal part of metatarsal 62.

A stabilizer 660 extends from distal end 640 and includes a pin 662 that can be inserted into a distal end of proximal part of metatarsal 62 or placed over top proximal part of metatarsal 62 to stabilize guide 600.

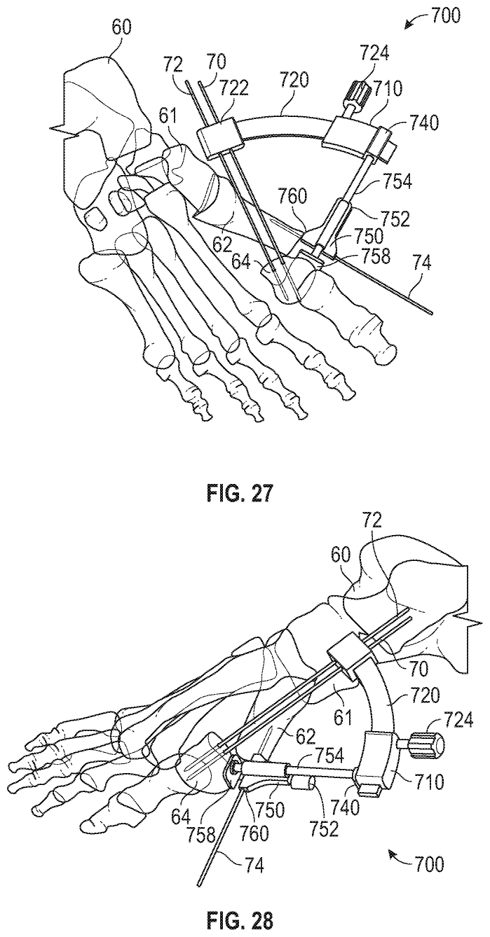

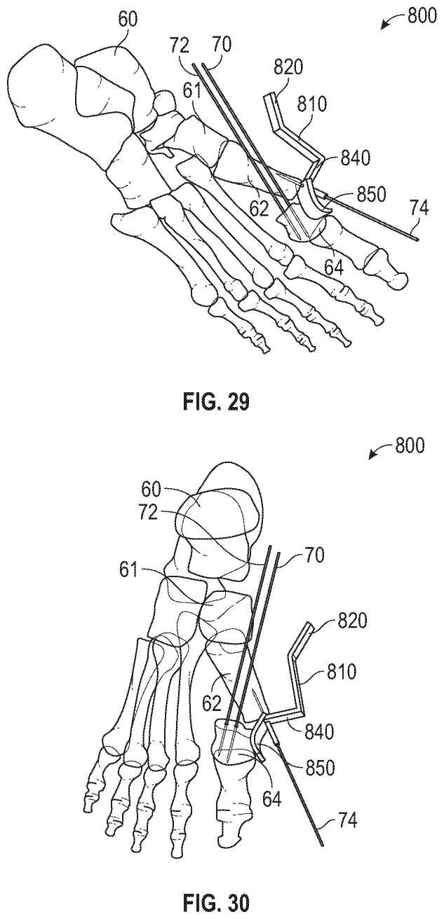

Referring to FIGS. 27 and 28, a metatarsal screw guide 700 (“guide 700”) according to another exemplary embodiment of the invention is shown. Guide 700 is used to align and connect proximal part of metatarsal 62 and distal part of the metatarsal 64 on foot 60 after a procedure, such as a bunionectomy. Guide 700 has a body 710 and an adjustable arcuate proximal K-wire guide 720 having a sleeve 722 that guides at least two K-wires 70, 72 into distal part of metatarsal 64. A locking knob 724 can be rotated to releasably lock K-wire guide 720 relative to body 710.

Body 710 has a distal end 740 having a pusher 750 with a threaded knob 752 having an internal threaded portion (not shown) that engages a complementary thread 754 on pusher 750 to extend or retract a pusher plate 758 to displace distal part of metatarsal 64 relative to proximal part of metatarsal 62.

A K-wire guide 760 is integrated into pusher 750 and allows for a K-wire 74 to be inserted into a distal end of proximal part of metatarsal 72.

Referring to FIGS. 29 and 30, a metatarsal screw guide 800 (“guide 800”) according to a fourth exemplary embodiment of the invention is shown. Guide 800 is used to align and connect proximal part of metatarsal 62 and distal part of the metatarsal 64 on foot 60 after a procedure, such as a bunionectomy. Guide 800 has a body 810 and a proximal pivot arm 820 extending proximally therefrom.

Body 810 has a distal end 840 having a pusher 850 with a K-wire guide 860 that is integrated into pusher 850 and allows for a K-wire 74 to be inserted into a distal end of proximal part of metatarsal 62. Pushing distally on proximal pivot arm 820 pushes pusher 850 downward, displacing distal part of metatarsal 64 away from proximal part of metatarsal 62 so that k-wire 74 can be inserted into a distal end of proximal part of metatarsal 62.

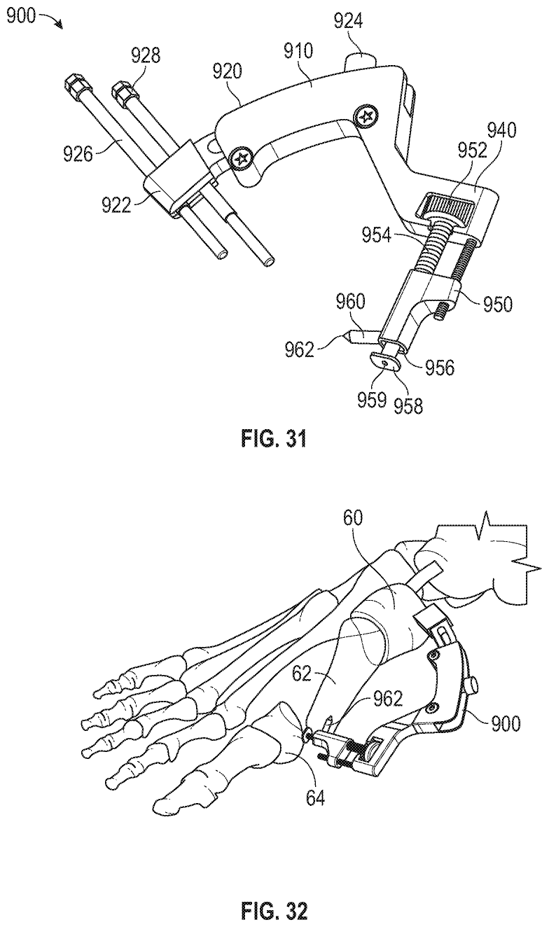

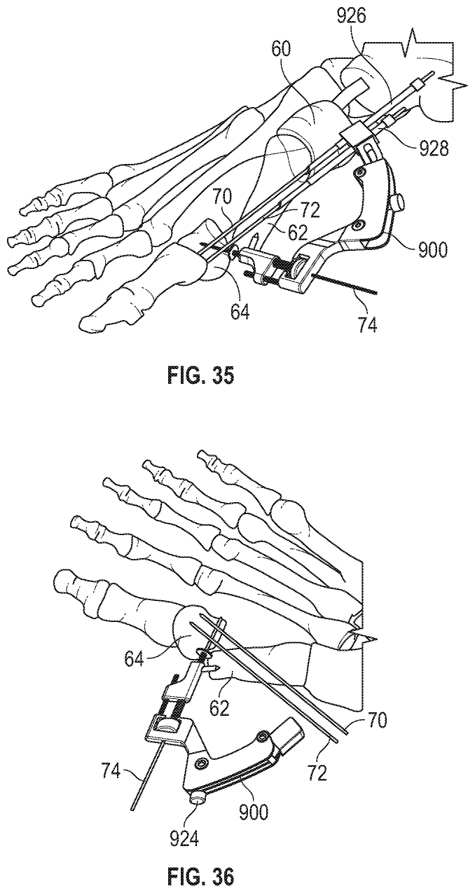

Referring to FIG. 31, a metatarsal screw guide 900 (“guide 900”) according to a fifth exemplary embodiment of the invention is shown. Guide 900 is used to align and connect proximal part of metatarsal 62 and distal part of the metatarsal 64 on foot 60 after a procedure, such as a bunionectomy. Guide 900 has an arcuate body 910 and a proximal K-wire guide 920 having an adjustable sleeve 922. K-wire guides 926, 928 extend from sleeve 922 and guide at least two K-wires 70, 72 (shown in FIG. 35) into distal part of metatarsal 64. A locking knob 924 can be rotated to releasably lock K-wire guide 920 relative to body 910.

Body 910 has a distal end 940 having a pusher 950 with a threaded reduction knob 952 having a threaded portion 954 that engages a complementary thread 956 on pusher 950 to extend or retract a pusher plate 958 to displace distal part of metatarsal 64 relative to proximal part of metatarsal 62. A third K-wire guide 959 extends the length of body 910 and exits pusher plate 958.

A stabilizer 960 extends from distal end 940 and includes a pin 962 that can be inserted into a distal end of proximal part of metatarsal 62 or placed over top proximal part of metatarsal 62 to stabilize guide 900.

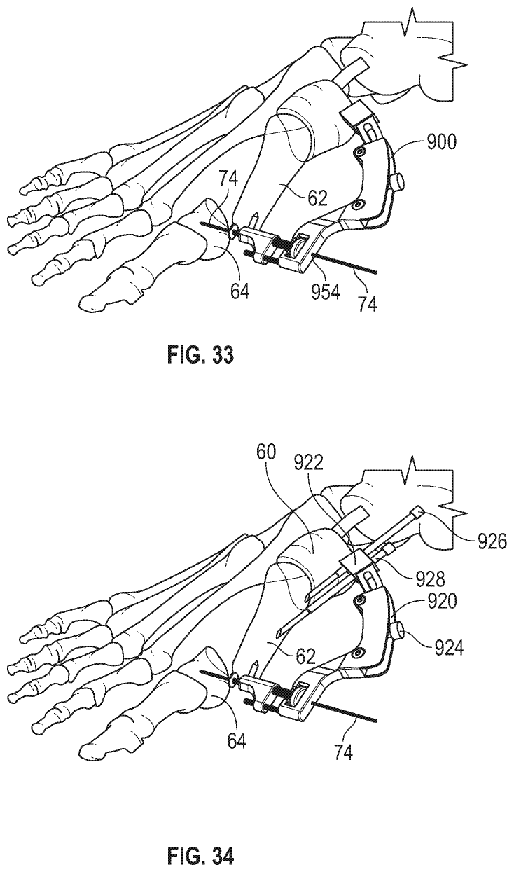

FIGS. 32-38 illustrate an exemplary method of securing proximal part of metatarsal 62 to distal part of metatarsal 64 in a procedure, such as a bunionectomy. FIG. 32 illustrates insertion of pin 962 into proximal part of metatarsal 62 and setting pusher plate 958 with knob 952. As shown in FIG. 33, a K-wire 74 is inserted through through-passage 959 and into distal portion of metatarsal 64.

FIG. 34 shows the insertion of K-wire guides 926, 928 through sleeve 922, proximal portion of metatarsal 62, and into distal portion of metatarsal 64, and setting and locking the arch of body 910 with knob 924. FIG. 35 shows the insertion of K-wires 70, 72 through K-wire guides 926, 928, respectively, with the distal tips of K-wires 70, 72 being inserted through proximal portion of metatarsal 62 and into the distal portion of metatarsal 64.

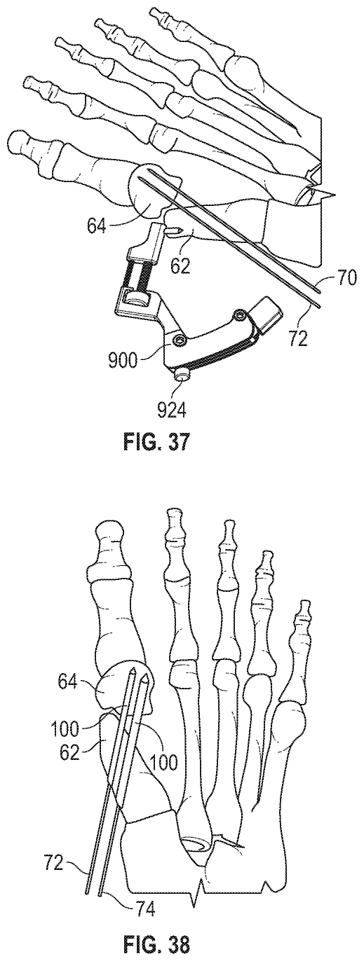

FIG. 36 shows guide 900 with K-wire guides removed and the reduction of guide 900 using knob 924. FIG. 37 shows guide 900 with K-wire 74 having been removed. Guide 900 is then removed from metatarsal portions 62, 64. FIG. 38 shows screws 100, having been inserted over K-wires 70, 72, and used to secure distal portion of metatarsal 64 to proximal portion of metatarsal 62.

It will be further understood that various changes in the details, materials, and arrangements of the parts which have been described and illustrated in order to explain the nature of this invention may be made by those skilled in the art without departing from the scope of the invention as expressed in the following claims.

1. A targeting guide comprising:
a body having a distal end, the distal end having a pusher with a threaded knob such that the threaded knob is rotatable to extend or retract a pusher plate; and
a first K-wire guide having a sleeve at a distal end thereof, the sleeve having at least two through-openings sized to allow the insertion of a K-wire through each of the at least two through-openings.

2. The targeting guide according to claim 1, wherein the first K-wire guide has an adjustable length.

3. The targeting guide according to claim 1, wherein the first K-wire guide has an arcuate arm.

4. The targeting guide according to claim 1, wherein the body further comprises a locking knob configured to releasably lock the first K-wire guide to the body.

5. The targeting guide according to claim 1, further comprising a stabilizer extending from the distal end of the K-wire guide.

6. The targeting guide according to claim 5, wherein the stabilizer includes a pin insertable into a metatarsal to stabilize the targeting guide.

7. The targeting guide according to claim 1, wherein the body further comprises a second K-wire guide.

8. The targeting guide according to claim 7, wherein the second K-wire guide extends perpendicular to the body.

9. The targeting guide according to claim 7, wherein the body further comprises a third K-wire guide.

10. The targeting guide according to claim 9, wherein the third K-wire guide extends along a length of the body.

11. A targeting guide comprising:
a body having:
a distal end;
a pusher plate located at the distal end; and
a pusher configured to extend or retract the pusher plate; and
a K-wire guide having a sleeve at a distal end of the K-wire guide, the sleeve having a through-opening sized to allow the insertion of a K-wire through the through-opening.

12. The targeting guide according to claim 11, further comprising a K-wire configured to extend through the through-opening.

13. The targeting guide according to claim 11, wherein the K-wire guide comprises a first K-wire guide and a second K-wire guide.

14. The targeting guide according to claim 11, wherein the K-wire comprises a first K-wire configured to extend through the first K-wire guide and a second K-wire configured to extend through the second K-wire guide.

15. The targeting guide according to claim 14, wherein the first and second K-wires are configured to be inserted into a distal part of a metatarsal.

16. The targeting guide according to claim 11, wherein the pusher plate is configured to displace a distal part of a metatarsal relative to a proximal part of the metatarsal.

17. The targeting guide according to claim 11, wherein the pusher comprises a threaded knob configured to be rotated to extend or retract the pusher plate.

18. The targeting guide according to claim 11, wherein the K-wire guide comprises an arcuate shape.

19. The targeting guide according to claim 11, further comprising a stabilizer at the distal end of the body.

20. The targeting guide according to claim 19, wherein the stabilizer comprises a pin configured to be inserted into a distal end of a proximal part of a metatarsal.
